(19) United States
(12) Patent Application Publication
(10) Pub. No.: US 2018/0207027 A1
(43) Pub. Date: Jul. 26, 2018

(54) HEAT EXCHANGE PAD FOR THERMAL TEMPERATURE MANAGEMENT

(71) Applicants: BOARD OF REGENTS, THE UNIVERSITY OF TEXAS SYSTEM, Austin, TX (US); Mercury Biomed, LLC, Lyndhurst, OH (US)

(72) Inventors: Kenneth R. Diller, Elgin, TX (US); Gary L. McGregor, Pflugerville, TX (US); Dale Wagner, McCordsville, IN (US); Jon Harvey, Pendleton, IN (US); Brad Pulver, Shaker Heights, OH (US); James Kuras, Macedonia, OH (US)

(21) Appl. No.: 15/879,634

(22) Filed: Jan. 25, 2018

Related U.S. Application Data
(60) Provisional application No. 62/450,130, filed on Jan. 25, 2017.

(51) Int. Cl. A61F 7/02; A61F 7/00

(52) U.S. Cl. CPC A61F 7/02; A61F 7/0085; A61F 2007/0228; A61F 2007/0268; A61F 2007/0054

(57) ABSTRACT

Example heat exchange pads are described herein. A heat exchange pad can include an input compartment, an output compartment arranged in fluid connection with the input compartment, and an internal member disposed between the input and output compartments. The internal member can include an array of holes formed therein. Additionally, the internal member can be configured to produce impinging flow convection heat transfer in proximity to a heat exchange surface of the output compartment.

### CROSS-REFERENCE TO RELATED APPLICATIONS

This application claims the benefit of U.S. Provisional Patent Application No. 62/450,130, filed on Jan. 25, 2017, entitled “HEAT EXCHANGE PAD FOR THERMAL TEMPERATURE MANAGEMENT,” the disclosure of which is expressly incorporated herein by reference in its entirety.

### BACKGROUND

The field of thermal therapy includes applying controlled temperatures to superficial tissues for enhanced healing of soft tissues or managing body core temperature to achieve perioperative normothermia or therapeutic hypothermia in response to major organ or brain ischemia or to keep patients from becoming hypothermic during surgery under anesthesia. The most widely used technology for this therapy is the application of water perfusion pads to a portion of a patient's skin. These pads are typically fabricated from a flexible polymeric material that is formed to embody internal flow channels through which water at a specified temperature is pumped with a velocity that is parallel to the active heat transfer surface (e.g., the pad-patient interface). Conventional pads use the same basic design, with small variations for the geometric pattern, number, and diameter of flow channels, and the overall shape. Conventional pads attempt to match a targeted anatomic location on the patient's body. For example, the pads can vary greatly in size from small (e.g., a few inches square) to large enough to cover an entire limb or torso of the patient. The pads can also vary greatly in the uniformity of temperature produced on the skin surface (or in many cases, the lack thereof). This heterogeneity in performance has been measured and documented, for example, in Khoshnevis, S., Nordhauser, J. E., Craik, N. K. and Diller, K. R., Quantitative Evaluation of the Thermal Heterogeneity on the Surface of Cryotherapy Cooling Pads, Journal of Biomechanical Engineering 136, 2014, 074503, 1-7.

Water perfusion pad designs incorporate internal architecture in the form of a channel, i.e., serpentine pathway; or create a reservoir, i.e., bladder in which the water flows into or through, thus providing a continuous temperature along the pad-patient interface. In conventional pads, the water flow channels are quite discrete in configuration and occupy only specific areas on the pad surface. Areas of the pad between the flow channels do not receive active temperature management. As a result, temperature management capability is compromised from a target value. The temperature pattern is more pronounced on some pad designs than on others. For example, in some pad designs, the active temperature control area is well below 50% of the total treatment area. On the best pads, active temperature control area can be half the total area.

Water flow channels function to force water to flow over as much of the treatment area as possible. Without water flow channels, e.g., in an open bladder configuration, water flow would take the path of least resistance, and omit temperature management for a large fraction of the treatment area. Some pad designs divide the flow into parallel channels in an attempt to bring fresh water to as much of the treatment area as possible, but unequal flow resistance may develop because of heterogeneous pressure loading on the pad, causing differentials in local temperature management levels. Alternatively, some pad designs embody a serpentine serial flow pattern in which the same water reaches all areas covered by the flow channel. The serpentine design causes forced (and typically) laminar flow controlled by a narrow path in which the temperature is constantly changing from the input to output, thus creating a very uneven temperature along the pad-patient interface. A major drawback of the serpentine design is that the water continuously changes temperature as it flows through the pad, resulting in uneven treatment along the pad-patient interface. The further the water flows through the pad, the closer its temperature approaches that of the underlying tissue, and the less effective the heat transfer becomes. In other words, a longer serpentine channel results in a greater temperature delta along the pathway.

Additionally, in some pad designs, water flow channels protrude from the base material, especially when pads are filled with pressurized water, further reducing the effective area to deliver a heating or cooling effect to a treatment site. Pads are typically affixed to a treatment site by straps to hold the pads in position. When a pad is placed into position and water flow is initiated, such a pad swells against the resistance of the holding straps, causing flexible water flow channels to be squeezed. In some cases, the flow distribution is altered to areas where the flow channels are not compressed, further exacerbating heterogeneity of the temperature pattern produced at the treatment site.

Further, in some pad designs, the pads do not conform easily to the morphological contours of the body, especially when the shapes are complex and involve small radii of curvature. The result is air gaps between the pad surface and skin surface that cause large local resistances to heat transfer and uneven temperature patterns on the skin.

### SUMMARY

Example heat exchange pads are described herein. A heat exchange pad can include an input compartment, an output compartment arranged in fluid connection with the input compartment, and an internal member disposed between the input and output compartments. The internal member can include an array of holes formed therein. This disclosure contemplates that the holes can be openings formed in the internal member. Additionally, the internal member can be configured to produce impinging flow convection heat transfer in proximity to a heat exchange surface of the output compartment.

Additionally, the internal member can be further configured to produce flow having a velocity vector primarily perpendicular to the heat exchange surface. Additionally, the internal member can optionally be further configured to produce flow having impingement onto the heat exchange surface.

Alternatively or additionally, the internal member can be further configured to produce a uniform flow pattern through the input compartment and to create a well-mixed flow pattern in the output compartment.

Alternatively or additionally, the input and output compartments can be formed from a plurality of layers including an input membrane, an output membrane, and the internal member. The internal member can be disposed between the input and output membranes. Alternatively or additionally, the output membrane can optionally be configured to interface with a patient's skin.

Alternatively or additionally, the input membrane can be a thermal insulator. Alternatively or additionally, the output membrane can be a thermal conductor. Alternatively or additionally, the input membrane and/or the internal member can be a semi-rigid or rigid material. Alternatively or additionally, the output membrane can be a compliant material.

Alternatively or additionally, the input membrane, the output membrane, and the internal member can be heat or pressure welded around a perimeter thereof.

Alternatively or additionally, the heat exchange pad can further include at least one tether member configured to couple the internal member and at least one of the input membrane or the output membrane.

Alternatively or additionally, the output membrane can be configured to removably couple to the internal member. Optionally, the input membrane can be coupled to the internal member such that the input membrane and the internal member form a reusable cartridge. Alternatively or additionally, the output membrane can optionally be disposable.

Alternatively or additionally, a number or arrangement of holes in the array of holes can be selected according to a total number of the holes, relative sizes of the holes, respective shapes of the holes, and/or a geometric pattern of the holes to produce a substantially uniform flow pattern through the internal member with minimal pressure drop so as to maximize an impingement flow convection effect at the output membrane.

Alternatively or additionally, a number or arrangement of holes in the array of holes can be selected to maximize a heat flux density across the heat exchange surface.

Alternatively or additionally, the heat exchange surface can be configured to interface with a patient's skin.

Alternatively or additionally, the internal member can be configured to increase a rate of heat transfer between fluid flowing through the output chamber and the patient's skin.

Alternatively or additionally, the internal member can be configured to minimize spatial temperature variations at the interface with the patient's skin.

Other systems, methods, features and/or advantages will be or may become apparent to one with skill in the art upon examination of the following drawings and detailed description. It is intended that all such additional systems, methods, features and/or advantages be included within this description and be protected by the accompanying claims.

### BRIEF DESCRIPTION OF THE DRAWINGS

The components in the drawings are not necessarily to scale relative to each other. Like reference numerals designate corresponding parts throughout the several views.

FIG. 1 illustrates an example heat exchange pad according to implementations described herein.

FIG. 2 illustrates another example heat exchange pad according to implementations described herein.

FIGS. 3A-3C illustrate computational fluid dynamic (CFD) analyses for example water perfusion pads. FIG. 3A illustrates the CFD analysis for a 2-layer serpentine pad. In FIG. 3A, Total Energy was 76.59 Watts (W), Standard Deviation was 1.77° C., and Mean Temperature was 39.08° C. FIG. 3B illustrates the CFD analysis for a 3-layer impingement pad (e.g., a water perfusion pad). In FIG. 3B, Total Energy was 75.03 W, Standard Deviation was 2.07° C., and Mean Temperature was 39.38° C. FIG. 3C illustrates the CFD analysis for another 3-layer impingement pad. In FIG. 3C, Total Energy was 77.88 W, Standard Deviation was 1.98° C., and Mean Temperature was 39.72° C.

FIGS. 4A-4D illustrate the differential heat transfer performance between conventional parallel flow pads and a prototype water perfusion pad. FIGS. 4A and 4B illustrate a conventional parallel flow pad and its CFD simulation of heat flux distribution across the pad surface, respectively. The parallel flow pad of FIGS. 4A and 4B has a divider in the middle of a single compartment. FIGS. 4C and 4D illustrate the prototype water perfusion pad having an array of 166 holes and its CFD simulation of heat flux distribution across the pad surface, respectively. The prototype water perfusion pad of FIGS. 4C and 4D has an internal member with an array of 166 holes.

FIGS. 5A-5C illustrate an example heat exchange pad with at least one tether member according to implementations described herein. FIG. 5A is a side view of the example heat exchange pad. FIG. 5B is a perspective view of the example heat exchange pad (without the output membrane to show the inside of the output compartment). FIG. 5C is another perspective view of the example heat exchange pad (without the output membrane to show the inside of the output compartment).

### DETAILED DESCRIPTION

Unless defined otherwise, all technical and scientific terms used herein have the same meaning as commonly understood by one of ordinary skill in the art. Methods and materials similar or equivalent to those described herein can be used in the practice or testing of the present disclosure. As used in the specification, and in the appended claims, the singular forms “a,” “an,” “the” include plural referents unless the context clearly dictates otherwise. The term “comprising” and variations thereof as used herein is used synonymously with the term “including” and variations thereof and are open, non-limiting terms. The terms “optional” or “optionally” used herein mean that the subsequently described feature, event or circumstance may or may not occur, and that the description includes instances where said feature, event or circumstance occurs and instances where it does not. Ranges may be expressed herein as from “about” one particular value, and/or to “about” another particular value. When such a range is expressed, an aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. While implementations will be described for water perfusion pads, it will become evident to those skilled in the art that the implementations are not limited thereto, but are also applicable for heat exchange pads using other fluids. The heat exchange pads described herein can be used, for example, in occupational, military, and/or athletic applications.

Heat exchange pads such as water perfusion pads are described herein. A water perfusion pad is a heat exchanger for the human body. The pad can be powered by a controller that heats or cools fluid (e.g., water) and uses a pump to force the fluid through and into the pad in a circulatory manner. The pad can provide either a cooling or heating surface to specific regions of the body for therapeutic purposes. The water perfusion pads described herein overcome issues associated with conventional pads described above by creating a well-mixed flow directed perpendicular to the pad-patient interface. This results in a more efficient heat transfer and uniform temperature at the pad-patient interface. The primary therapeutic outcomes of the water perfusion pads described herein are an enhanced level of heat transfer per unit pad area and a more uniform temperature distribution over the treatment area. Conventional pads are deficient in one or both of these areas.

The example water perfusion pads described herein include two chambers (e.g., input and output compartments) bridged by a middle barrier (e.g., an internal member) having holes (or ovals, slits, or other openings) scattered along its planar or curved surface. A first chamber (e.g., the input compartment) can be formed of a rigid material (e.g., polycarbonate), or alternatively, a flexible material (e.g., polyvinyl chloride (PVC)), creating a hollow chamber into which fluid is fed, for example, through an input nozzle. The first chamber can accommodate a pool of fluid that is pressurized to maintain a flow rate through the flow restriction caused by the middle barrier. The pressure is sufficient to force the fluid to flow through the holes of the middle barrier and into a second chamber (e.g., an output compartment). The second chamber can be formed of a compliant material (e.g., PVC), which acts as the pad-patient interface. As the fluid flows through the holes of the middle barrier, the velocity of the fluid increases, and the fluid impinges along the pad-patient interface, which creates a well-mixed environment homogenizing the temperature within the second chamber and minimizing the convective heat flow resistance with the output membrane. This results in an increased heat exchange along the pad-interface. The fluid can exit the pad through an outlet nozzle.

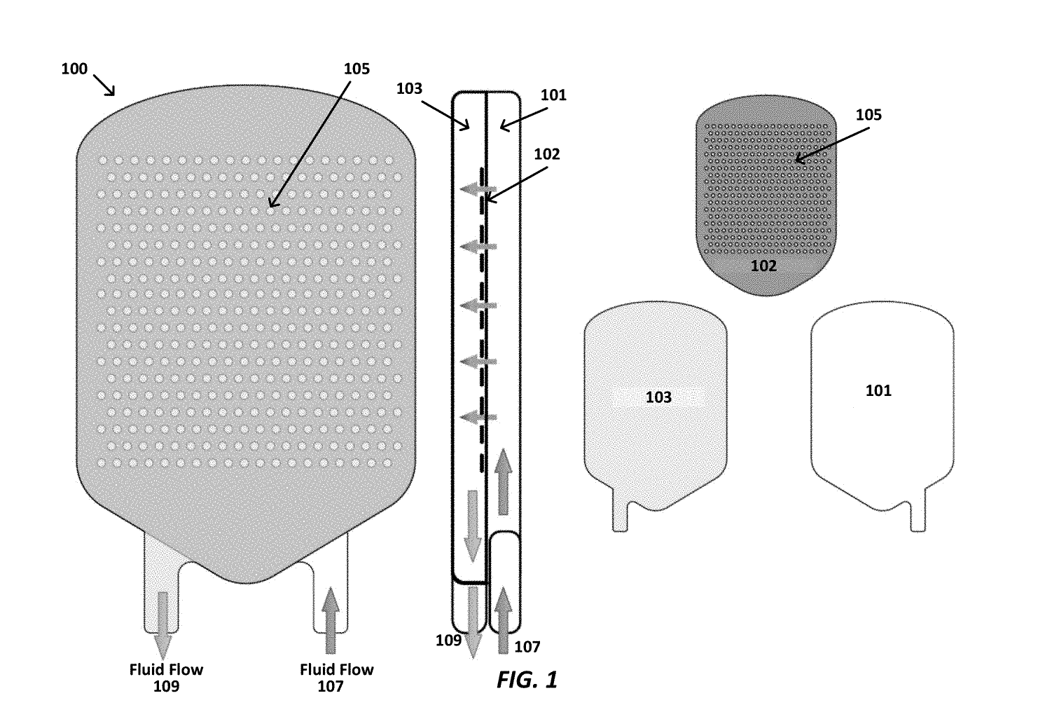

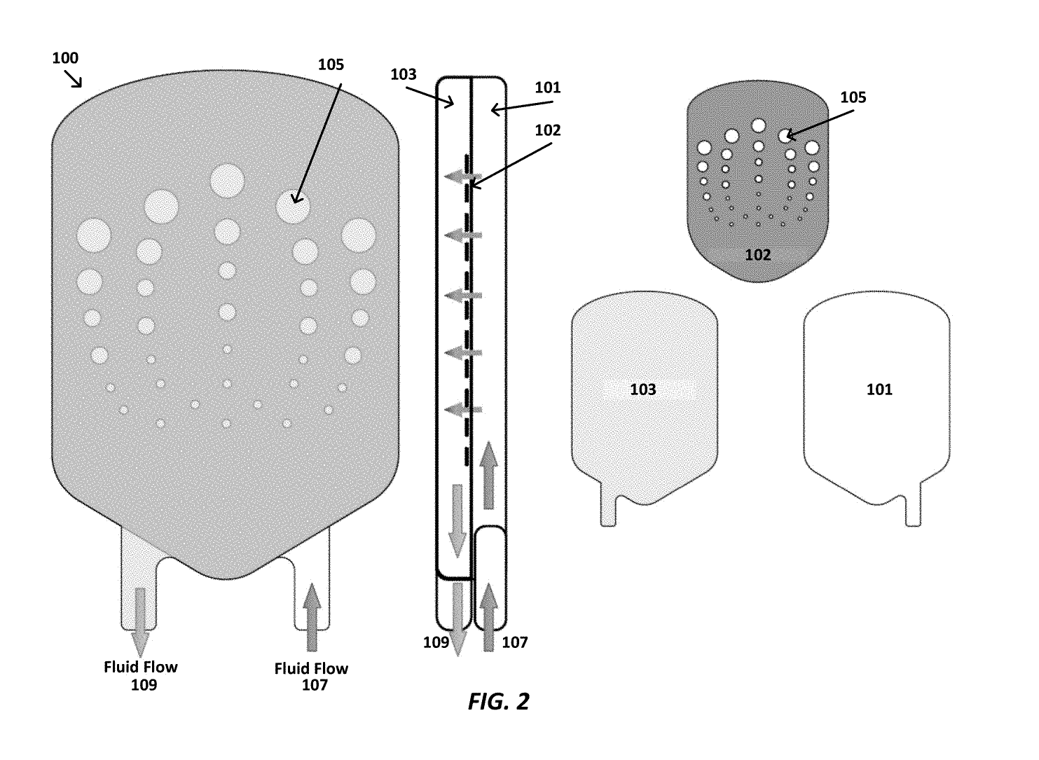

Referring now to FIGS. 1 and 2, example heat exchange pads are shown. The heat exchange pad 100 can include an input compartment 101, an output compartment 103 arranged in fluid connection with the input compartment 101, and an internal member 102 disposed between the input compartment 101 and output compartment 103. When applied to the patient's body, the input compartment 101 can be arranged away from the patient's skin, and the output compartment 103 can be arranged adjacent to the patient's skin. As described below, a portion of the output compartment 103 can act as the pad-patient interface. The internal member 102 can include an array of holes 105 formed therein. The input compartment 101 can be a hollow chamber that can accommodate a pool of fluid, which enters the input compartment through an input nozzle or opening 107. As fluid pressure increases, the fluid is forced through the holes of the internal member 102 and into the output compartment 103. The fluid can exit the output compartment 103 though an output nozzle or opening 109.

The internal member 102 can be configured to produce impinging flow convection heat transfer in proximity to a heat exchange surface (e.g., the heat exchange pad-patient interface) of the output compartment 103. Optionally, the internal member 102 can be configured to produce flow having a velocity vector perpendicular to the heat exchange surface. Additionally, the internal member can optionally be further configured to produce flow having impingement onto the heat exchange surface. In other words, the flow moving with a velocity vector primarily perpendicular to the heat exchange surface can contact the heat exchange surface (e.g., be impingement upon) with a momentum that enhances convection efficacy. Alternatively or additionally, the internal member 102 can optionally be configured to produce a uniform flow pattern through the input compartment 101 and to create a well-mixed flow pattern in the output compartment 103. This results in mixing of the fluid within the output compartment 103 and a more even temperature distribution and convective heat transfer over the pad-patient interface. Optionally, the well-mixed flow pattern can be a turbulent flow pattern. For example, a number or arrangement of holes in the array of holes 105 can be selected according to a total number of the holes, relative sizes of the holes, respective shapes of the holes, and/or a geometric pattern of the holes to produce a substantially uniform flow pattern through the internal member 102 with minimal pressure drop so as to maximize an impingement flow convection effect at the pad-patient interface. Alternatively or additionally, a number or arrangement of holes in the array of holes 105 can be selected to maximize a heat flux density across the heat exchange surface. Alternatively or additionally, the internal member 102 can be configured to increase a rate of heat transfer between fluid flowing through the output compartment 103 and the patient's skin. Alternatively or additionally, the internal member 102 can be configured to minimize spatial temperature variations at the interface with the patient's skin.

This disclosure contemplates that a number, size, and/or arrangement of holes in the array of holes 105 can be selected to achieve the above effects. This disclosure also contemplates that the holes of the array of holes 105 can be openings formed in the internal member 102. Additionally, this disclosure contemplates that the holes of the array of holes 105 can be circular, ovals, slits, and/or openings having other geometries. It should be understood that the number, size, arrangement, etc. of the holes of the array of holes 105 shown in FIGS. 1 and 2 are provided only as examples. For example, the number, pattern, size, size distribution, shape of the holes, etc. can be selected to match specific applications that can be a function of one or more application-specific factors including, but not limited to, coverage area, anatomical site, therapeutic need, etc. Accordingly, this disclosure contemplates that the number, size, shape, and/or arrangement of the holes of the array of holes 105 can be different than those shown in FIGS. 1 and 2.

In some implementations, the input compartment 101 and output compartment 103 can be formed from a plurality of layers including an input membrane, an output membrane, and the internal member 102. The internal member 102 can be disposed between the input and output membranes. For example, the input membrane, the output membrane, and the internal member 102 can optionally be heat or pressure welded around a perimeter thereof. The input membrane and/or the internal member 102 can optionally be a semi-rigid or rigid material. Example semi-rigid or rigid materials include, but are not limited to, thermoplastics, polycarbonate, ABS, and/or metal. Alternatively or additionally, the input membrane can optionally be a thermal insulator. Example thermal insulators include, but are not limited to, open or closed cell silicone, polyvinyl chloride (PVC), or polyurethane sponge or foam. Alternatively or additionally, the output membrane can optionally be configured to interface with a patient's skin. In other words, the output membrane can form the pad-patient interface. Accordingly, the output membrane can optionally be a thermal conductor. Example thermal conductors include, but are not limited to, polyurethane film, closed cell thermally conductive silicone sponge, graphite sheeting and/or polyvinyl chloride (PVC). Alternatively or additionally, the output membrane can optionally be a compliant material. Example compliant materials include, but are not limited to, silicone and polyurethane foams and films.

In some implementations, the output membrane can be configured to removably couple to the internal member 102. Optionally, the input membrane can be coupled to the internal member 102 such that the input membrane and the internal member 102 form a reusable cartridge. Alternatively or additionally, the output membrane can optionally be disposable. Alternatively or additionally, the output membrane can have an external cover that can optionally be disposable.

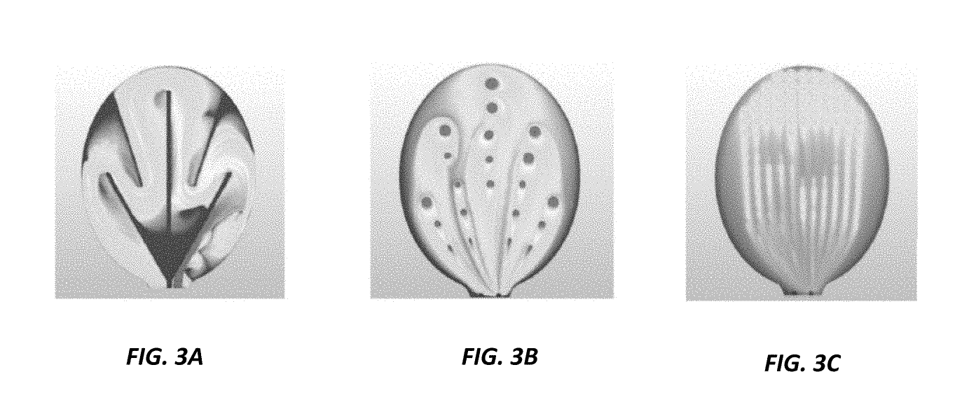

Referring now to FIGS. 3A-3C, respective CFD analyses for three example water perfusion pads are shown. FIG. 3A illustrates the CFD analysis for a 2-layer serpentine pad. FIG. 3B illustrates the CFD analysis for a 3-layer impingement pad according to implementations described herein. FIG. 3C illustrates the CFD analysis for another 3-layer impingement pad according to implementations described herein. The CFD analysis predicts the total energy transfer along that heat exchange pad-patient interface. The total energy, standard temperature deviation, and mean temperature are shown below each of FIGS. 3A-3C.

### EXAMPLES

The heat exchange pads described herein enhance the effectiveness of heat transfer between fluid (e.g., water) flowing through the pad and the surface of human skin onto which it is placed. The pads embody advanced heat exchanger design principles to achieve, among other, two objectives: (1) to produce a larger rate of heat transfer per unit area (W/m²) between water flowing through the pad and human tissue onto which it is placed; and (2) to produce on the skin surface a temperature pattern that has minimal spatial variations so as to produce superior therapeutic outcomes.

The heat exchange pad can has two water flow compartments (e.g., input and output compartments 101 and 103 in FIGS. 1 and 2) that act in concert to focus the heat transfer onto a surface that faces the body tissue to which therapy is applied (e.g., the heat exchange surface and/or pad-patient interface). The two compartments can be formed from three layers of materials of approximately the same size and shapes and that are welded or sealed together around their perimeters.

All of the materials can be sheets of a deformable polymer, each of which may have a unique combination of thermal and mechanical properties that contribute to effective functioning of the heat exchanger.

The two compartments can be designed so that water flows in a perpendicular manner onto the heat exchange surface that is facing the therapeutic area of the body, producing impinging flow convection heat transfer, which is the most thermally effective means of convection. As described above, conventional therapeutic pads are typically based on parallel flow convection heat transfer in which the momentum of moving water is along rather than against the heat exchange surface. Parallel flow is far less effective for convection than is impinging flow.

The inlet flow compartment (e.g., input compartment 101 in FIGS. 1 and 2) can be arranged away from the body surface, and the outlet flow compartment (e.g., output compartment 103 in FIGS. 1 and 2) can be arranged adjacent to the body surface.

The two compartments can be separated by an internal member (e.g., internal member 102 in FIGS. 1 and 2) having an array of holes (e.g., array of holes 105 in FIGS. 1 and 2) designed for their relative size distribution and relative spatial placement to produce a largely uniform flow pattern between the inlet and outlet flow compartments. The number of holes may be adjusted depending on the heat transfer and water flow requirements. An example prototype water perfusion pad includes an internal member with an array of 166 holes of 1/16 inch diameter.

The flow leaving the inlet flow compartment and entering the outlet flow compartment can be forced to have a velocity vector perpendicular to the surface of the outlet flow compartment that is proximal to the body surface, producing an impingement convection effect on that surface, thereby maximizing the heat transfer between the flowing controlled temperature water and the aspect of the heat exchanger that acts on the body part.

The pattern of separation distance between adjacent holes can be designed such that the impingement convection effect for each hole is individually maximized without being compromised by flow influences from adjacent holes, but while achieving the highest density of heat flux across the entire surface that acts on the body part.

The impingement flow heat transfer pad can be scaled across various sizes to accommodate different therapeutic locations on the body surface.

The pad can be fabricated with different materials selected for their thermal and mechanical performance properties. For example, the outer membrane of the pad that is positioned away from the tissue treatment area can be fabricated from a material having thermal insulating properties to reduce heat exchange with the environment. The inner membrane of the pad that is positioned against the tissue treatment area can be desirably fabricated of a highly conductive material to enable uniform distribution of temperature over the treatment site. The conductive surface acts to spread the temperature and reduce gradients on the treatment site.

The three layers of the two compartment pad can be sealed or heat pressure welded around their perimeter.

The internal member with the array of holes can be made of thicker stock to enhance mechanical stability

The entire pad can be flexible to be able to conform to the shape of a body area to which it is applied

The inlet flow chamber can be positioned away from the body therapeutic surface and acts as a collecting volume for water at the treatment temperature and that is at an elevated pressure that forces water to flow with a high momentum through the holes in the interior layer and then be perpendicularly impingent onto the active heat transfer surface that contacts the treatment tissue. The inlet flow chamber can be insulated on its exterior surface so that all of the water is nearly isothermal prior to impinging on the active heat transfer surface, thereby producing a nearly uniform temperature on the treatment surface.

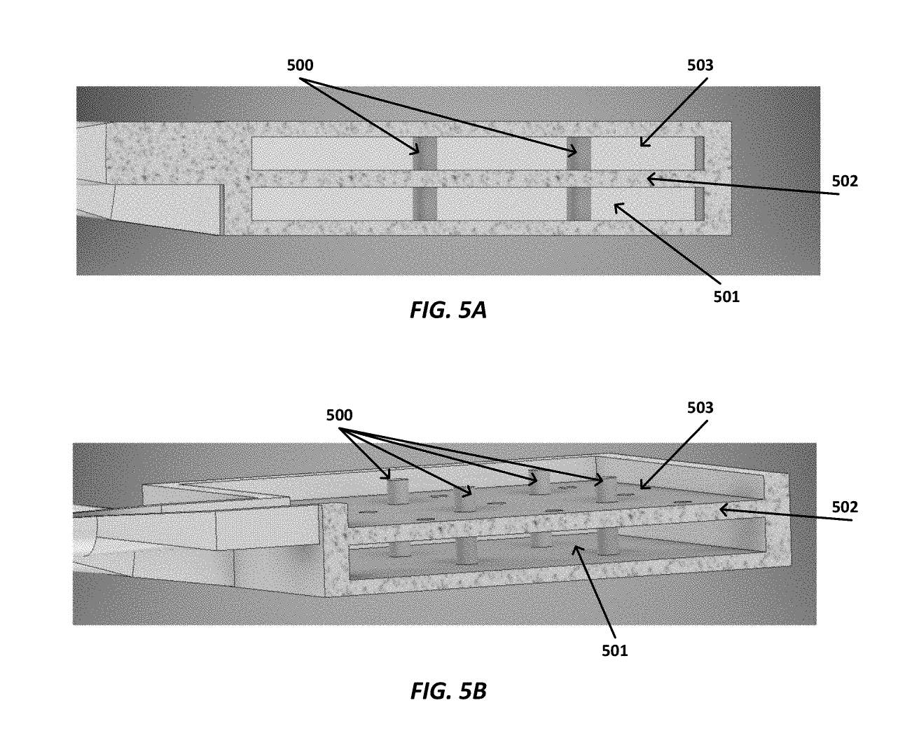

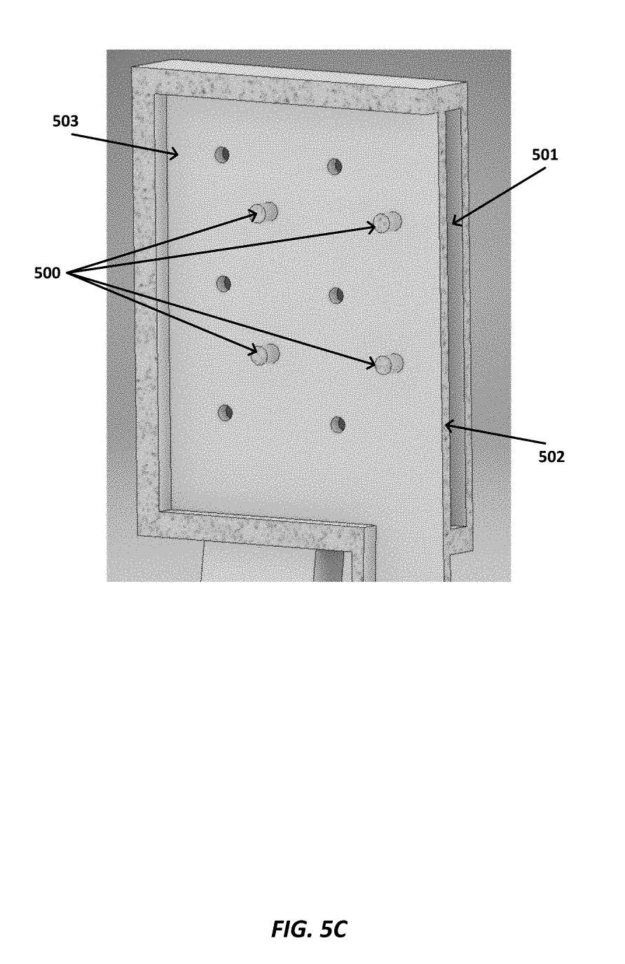

Since the inlet flow chamber has the highest pressure interior to the heat exchanger pad and it is also flexible to be able to conform to body contours, the inlet flow chamber can tend to expand and assume a rounded shape in use. This effect can be limited by fabricating the heat exchanger with multiple internal tether elements (e.g., one or more tether members coupling the internal member 102 and at least one of the input membrane or output membrane of FIGS. 1 and 2) that are anchored solidly to the two exterior pad surfaces. The tether anchor points can be reinforced for tensile strength by an integral locally thicker material that acts as a washer. The tethers and anchors are desirably formed from polymer material similar to the other components of the heat exchanger pad. The tether members can prevent the heat exchanger from deforming when under pressure. FIGS. 5A-5C illustrate example tether members 500. FIGS. 5A-5C show the input compartment 501, internal member 502, and output compartment 503. It should be understood that the tether members 500 shown in FIGS. 5A-5C are provided only as an example. This disclosure contemplates using different types, numbers, and/or arrangements of tether members.

The momentum of water flowing through the holes in the internal member from the inlet flow chamber to the outlet flow chamber can tend to maintain an open flow channel for water moving through the outlet flow chamber from which heat transfer action is applied to the skin.

As described above, parallel flow water heat exchange pads consist of discrete flow channels that inflate and take on a rounded shape when pressurized water is forced through them, resulting in a nonplanar surface that does not make uniform contact with a treatment tissue. In contrast, because the water flow vector in the impingement flow heat exchange pads is perpendicular the active heat transfer surface and is distributed uniformly over that surface, the flow of water does not deform the pad. The result is a uniform contact with an entire tissue therapy area, enabling a large net heat transfer with the tissue per unit treatment area.

Exterior straps can be used to further stabilize the pad to the treatment site.

Initial prototype heat exchanger pads have been constructed and tested, demonstrating superior heat exchange density and surface temperature uniformity to traditional parallel flow water heat exchange pads.

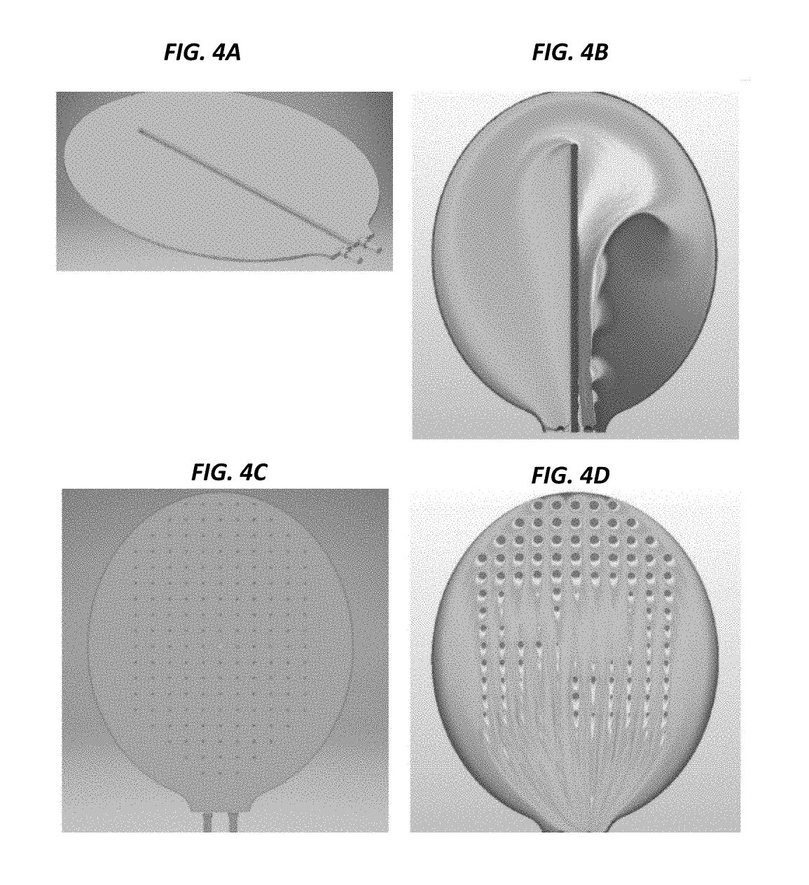

Referring now to FIGS. 4A-4D, the differential heat transfer performance between conventional parallel flow pads and a prototype water perfusion pad are shown. FIGS. 4A and 4B illustrate a conventional parallel flow pad and its CFD simulation of heat flux distribution across the pad surface, respectively. FIGS. 4C and 4D illustrate the prototype water perfusion pad having an array of 166 holes and its CFD simulation of heat flux distribution across the pad surface, respectively.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

1. A heat exchange pad, comprising:
an input compartment;
an output compartment arranged in fluid connection with the input compartment; and
an internal member disposed between the input and output compartments, wherein the internal member comprises an array of holes formed therein, and wherein the internal member is configured to produce impinging flow convection heat transfer in proximity to a heat exchange surface of the output compartment.

2. The heat exchange pad of claim 1, wherein the internal member is further configured to produce flow having a velocity vector perpendicular to the heat exchange surface.

3. The heat exchange pad of claim 1, wherein the internal member is further configured to produce flow having impingement onto the heat exchange surface.

4. The heat exchange pad of claim 1, wherein the internal member is further configured to produce a uniform flow pattern through the input compartment and to create a well-mixed flow pattern in the output compartment.

5. The heat exchange pad of claim 1, wherein the input and output compartments are formed from a plurality of layers comprising an input membrane, an output membrane, and the internal member, the internal member being disposed between the input and output membranes.

6. The heat exchange pad of claim 5, wherein the output membrane is configured to interface with a patient's skin.

7. The heat exchange pad of claim 5, wherein the input membrane comprises a thermal insulator.

8. The heat exchange pad of claim 5, wherein the output membrane comprises a thermal conductor.

9. The heat exchange pad of claim 5, wherein at least one of the input membrane or the internal member comprises a semi-rigid or rigid material.

10. The heat exchange pad of claim 5, wherein the output membrane comprises a compliant material.

11. The heat exchange pad of claim 5, wherein the input membrane, the output membrane, and the internal member are heat or pressure welded around a perimeter thereof.

12. The heat exchange pad of claim 5, further comprising at least one tether member configured to couple the internal member and at least one of the input membrane or the output membrane.

13. The heat exchange pad of claim 5, wherein the output membrane is configured to removably couple to the internal member.

14. The heat exchange pad of claim 13, wherein the input membrane is coupled to the internal member, the input membrane and the internal member forming a reusable cartridge.

15. The heat exchange pad of claim 13, wherein the output membrane is disposable.

16. The heat exchange pad of claim 1, wherein a number or arrangement of holes in the array of holes is selected according to a total number of the holes, relative sizes of the holes, respective shapes of the holes, and/or a geometric pattern of the holes to produce a substantially uniform flow pattern through the internal member with minimal pressure drop so as to maximize an impingement flow convection effect at the output membrane.

17. The heat exchange pad of claim 1, wherein a number or arrangement of holes in the array of holes is selected to maximize a heat flux density across the heat exchange surface.

18. The heat exchange pad of claim 1, wherein the heat exchange surface is configured to interface with a patient's skin.

19. The heat exchange pad of claim 18, wherein the internal member is configured to increase a rate of heat transfer between fluid flowing through the output compartment and the patient's skin.

20. The heat exchange pad of claim 18, wherein the internal member is configured to minimize spatial temperature variations at the interface with the patient's skin.
